(19) United States
(12) Patent Application Publication
(10) Pub. No.: US 2021/0375468 A1
(43) Pub. Date: Dec. 2, 2021

(54) Using Electronic Health Records and Machine Learning to Predict and Mitigate Postpartum Depression

(71) Applicant: Cornell University, Ithaca, NY (US)

(72) Inventors: Yiye Zhang, New York, NY (US); Jyotishman Pathak, New York, NY (US); Shuojia Wang, New York, NY (US); Alison Herman, Brooklyn, NY (US); Rochelle Joly, Bronx, NY (US)

(21) Appl. No.: 17/331,469

(22) Filed: May 26, 2021

Related U.S. Application Data
(60) Provisional application No. 63/030,434, filed on May 27, 2020.

(51) Int. Cl. G16H 50/30; G06N 20/00; G16H 10/60; G16H 20/70; G16H 50/20; A61B 5/16

(52) U.S. Cl. CPC G16H 50/30; G06N 20/00; A61B 5/165; G16H 20/70; G16H 50/20; G16H 10/60

(57) ABSTRACT

A method includes receiving health data for a patient. Based on the health data for the patient, a risk score is computed, indicating the patient's risk of developing postpartum depression, as a function of features selected from the health data for the patient. If the risk score exceeds a threshold value, treatment recommendations are provided based on the health data for the patient. The health data may include anxiety history, use of antidepressants, mood disorder history, an indicator of whether there has been depression in pregnancy, an indicator of whether there has been anxiety in pregnancy, an indicator of whether there has been mental disorder in pregnancy, and a history of other disorders.

### CROSS REFERENCE TO RELATED APPLICATIONS

This application claims the priority benefit of U.S. Provisional Patent Application No. 63/030,434, filed 27 May 2020, hereby incorporated by reference in its entirety as though fully set forth herein.

### TECHNICAL FIELD

The subject matter described herein relates to devices, systems, and methods for assessing and mitigating the risk of postpartum depression. These postpartum prediction systems and methods have particular but not exclusive utility for medical diagnosis and treatment of pregnant women.

### BACKGROUND

Postpartum depression (PPD) is a nonpsychotic depressive episode that begins one year within childbirth, interfering with mothers' emotional wellbeing as well as the children's morbidity, cognitive skills, and behavioral skills later in life. Commonly used postpartum treatment methods only treat such depression after it has occurred, which may limit the timeliness and effectiveness of treatment. Accordingly, a need exists for improved treatment methods that address the forgoing and other concerns.

There are 2 United States Preventive Services Task Force statements relevant to this problem. The first (2016) is a level B recommendation for pregnant and postpartum women to be routinely screened for depression in primary care settings and referred for interventional treatment if positive. The second (2019) is a level B recommendation that pregnant women known to be at high risk for postpartum depression be referred for preventive psychotherapy. Both recommendations have been challenging to implement, although the first is being widely adopted in obstetric and pediatric practices. The second has not been widely adopted, due to the a lack of tools to systematically determine who is at risk.

The information included in this Background section of the specification, including any references cited herein and any description or discussion thereof, is included for technical reference purposes only and is not to be regarded as subject matter by which the scope of the disclosure is to be bound.

### SUMMARY

Postpartum depression (PPD) is one of the most frequent maternal morbidities after delivery with serious implications. Currently, there is a lack of effective screening strategies and high-quality clinical trials. The ability to leverage a large amount of detailed patient data from electronic health records (EHRs) to predict PPD could enable the implementation of effective clinical decision support interventions. To develop a PPD prediction model, using EHRs, 9,980 episodes of pregnancy was identified. Six machine learning algorithms, including L2-regularized Logistic Regression, Support Vector Machine, Decision Tree, Naïve Bayes, extreme gradient boosting, and Random forest were constructed. The model's best prediction performance achieved an AUC of 0.79. Race, obesity, anxiety, depression, different types of pain, antidepressants, and anti-inflammatory drugs during pregnancy were among the significant predictors. The results suggest a potential for applying machine learning to EHR data to predict PPD and inform healthcare delivery.

Disclosed are devices, systems, and methods for predicting and mitigating the occurrence of postpartum depression. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer implemented method. The computer implemented method includes, in a processor including a memory, performing the steps of: identifying a patient population including a plurality of members; collecting data from electronic health records of the plurality of members; for each member of the plurality of members, identifying whether or not the member developed postpartum depression; and training a machine learning algorithm to identify a patient's probability of developing postpartum depression using training data based on the collected data for the plurality of members. Other embodiments of this aspect include corresponding computer systems, apparatus, and computer programs recorded on one or more computer storage devices, each configured to perform the actions of the methods.

Implementations may include one or more of the following features. In some embodiments, the computer implemented method further includes ranking selected elements of the collected data according to their statistical correlation with a patient developing postpartum depression. In some embodiments, the computer implemented method further includes computing weights for each selected element of the ranked selected elements. In some embodiments, the computer implemented method further includes removing elements from the training data whose weights are below a threshold value. In some embodiments, the selected elements of the collected data include socio-demographic data, medication data, lab data, emergency department visit data, marital status, mental health history, medical comorbidity diagnosis data, and obstetric complications. In some embodiments, the machine learning algorithm includes at least one of a logistics regression, support vector machine, naive Bayes, random forest, decision tree, extreme gradient boosting, or multi-layer perceptron classifier. In some embodiments, when trained with the collected data, the machine learning algorithm has a Brier score of no more than 0.181 or an area under a receiver operating characteristic (ROC) curve of no less than 0.886. Implementations of the described techniques may include hardware, a method or process, or computer software on a computer-accessible medium.

One general aspect includes a computer implemented method including, in a processor including a memory, performing the steps of: receiving health data for a patient; based on the health data for the patient, computing a risk score of the patient's risk of developing postpartum depression as a function of features selected from the health data for the patient; if the risk score exceeds a threshold value, computing treatment recommendations based on the health data for the patient, and providing the treatment recommendations. Other embodiments of this aspect include corresponding computer systems, apparatus, and computer programs recorded on one or more computer storage devices, each configured to perform the actions of the methods.

Implementations may include one or more of the following features. In some embodiments, computing the risk score involves receiving at least some of the health data for the patient into a machine learning algorithm trained using health data from a population of pregnant women. In some embodiments, the machine learning algorithm includes at least one of a logistics regression, support vector machine, naive Bayes, random forest, decision tree, extreme gradient boosting, or multi-layer perceptron classifier. In some embodiments, the population of pregnant women includes at least some health or demographic characteristics in common with the patient. In some embodiments, the risk score includes a numerical value between 0 and 1 or a percentage between 0% and 100%. In some embodiments, the computation of either the risk score or the treatment recommendations includes a function of features selected from the health data for the patient, where the features are selected from anxiety history, use of antidepressants, mood disorder history, an indicator of whether there has been depression in pregnancy, an indicator of whether there has been anxiety in pregnancy, an indicator of whether there has been mental disorder in pregnancy, and a history of other disorders. In some embodiments, the computation of either the risk score or the treatment recommendations includes a function of features selected from the health data for the patient, where the features are selected from palpitations, diarrhea, vomiting in pregnancy, hypertensive disorder, acute pharyngitis, hemorrhage in early pregnancy antepartum, patient's race is White, threatened miscarriage, abdominal pain, migraine, beta blocking agents, antihistamines for systemic use, hypothyroidism, placental infarct, patient's relationship status is single, deliveries by cesarean, direct acting antivirals, primigravida, pre-eclampsia, other antibacterials, number of emergency department visits, abnormality of organs and/or soft tissues of pelvis affecting pregnancy, diastolic blood pressure in third trimester, false labor at or after 37 completed weeks of gestation, or patient's race is Asian. In some embodiments, the patient's risk of developing postpartum depression includes a risk of a diagnosis of depression within 12 months after delivery. In some embodiments, the threshold value is 50%. In some embodiments, the threshold value is 75%. In some embodiments, the health data is received from at least one of an electronic health record, a wearable device, a handheld device, or a census database. In some embodiments, the processor is a point-of-care processor. In some embodiments, the patient is a pregnant woman. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the postpartum depression prediction method, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 shows the schematic diagram of an example PPD prediction framework that describes the various steps involved in data preprocessing and risk model development, in accordance with at least one embodiment of the present disclosure.

FIG. 2 shows the inclusion and exclusion criteria of this study, in accordance with at least one embodiment of the present disclosure.

FIG. 3 shows model performance for the model training data, in accordance with at least one embodiment of the present disclosure.

FIG. 4 shows model performance for the model validation data, in accordance with at least one embodiment of the present disclosure.

FIG. 5 shows a schematic in block diagram form of an example clinician workflow, in accordance with at least one embodiment of the present disclosure.

FIG. 6 shows an example clinical dashboard, in accordance with at least one embodiment of the present disclosure.

FIG. 7 shows a flow diagram of an example clinical workflow method in accordance with at least one embodiment of the present disclosure.

FIG. 8 is a schematic diagram of a processor circuit, according to embodiments of the present disclosure.

### DETAILED DESCRIPTION

Postpartum depression (PPD) is a nonpsychotic depressive episode that begins one year within childbirth. The prevalence of PPD is reported to be 13% in high-income countries and 15% in low and middle-income countries PPD is one of the most frequent and serious maternal morbidities after delivery. It not only interferes with mothers' emotional wellbeing, but is also associated with infant morbidity, and children's poorer cognitive and behavioral skills later in life.

Despite the serious adverse consequences of PPD, there is a lack of consensus and evidence on PPD screening and treatment from high-quality clinical trials. A number of key predictors have been identified from previous meta-analysis studies, including a history of psychiatric illness, prenatal depression, stressors and illness during pregnancy, poor social support, poor self-esteem, and lower socioeconomic status. Few studies found a significant association between prescription drug use during pregnancy with PPD.

Although risk factors were reported from previous studies, effective interventions against them and identification of at-risk women still need further evaluations. A number of screening and preventative measures were proposed previous studies with varying outcomes. For example, prior PPD prediction studies were prospective studies conducted with small sizes. Features used in these predictive models often included questionnaires measuring psychological statuses such as demographics, education level, self-esteem, and social support, but not current diagnoses and medications. A commonly used questionnaire for perinatal depression screening is the Edinburgh Postnatal Depression Scale (EPDS), although its effectiveness in screening has been questioned in previous studies.

The current knowledge gap on PPD contributes to substantial variations across clinical practices in screening and information collection. Addressing these challenges, it has been pointed out by the US Preventive Services Task Force (USPSTF) that electronic health records (EHRs)-based tools may be considered in implementing PPD-related interventions. EHR data may be routinely collected and contain a detailed history of health and health services utilization. Moreover, models developed using EHR data can be potentially integrated within the EHR system as clinical decision support (CDS), allowing effective screening for expectant mothers at risk of developing PPD.

Machine learning algorithms can be applied to EHR data, containing information from the full three trimesters of pregnancy period to delivery, to construct a predictive model for PPD outcome. In an example, a study is performed using six machine learning algorithms featuring longitudinal clinical information and patients' socio-demographic characteristics. Machine learning models can be used to predict PPD, and to carefully evaluate the risk factors identified from EHR data.

The present disclosure aids substantially in prediction, early detection, diagnosis, mitigation, and treatment of postpartum depression (PPD), by improving a clinician's ability to understand an individual patient's PPD risk at various times during pregnancy. Implemented on a processor in communication with data collection sensors and data compilation devices, the devices, systems, and methods disclosed herein provide practical medical intervention strategies. This improved clinical analysis transforms a clinician's “educated guesses” into a statistically rigorous set of risk factors, predictions and treatments, without the normally routine need for a clinician to pore through scattered electronic health records and other data. This unconventional approach improves the functioning of clinical point-of-care computing systems, by permitting them to compute PPD risk and output appropriate interventions.

The PPD prediction method may be implemented at least in part as a machine learning algorithm whose inputs and outputs are viewable on a display, and which may be operated by a control process executing on a processor that accepts user inputs from a keyboard, mouse, or touchscreen interface, and that is in communication with one or more databases and/or sensing devices. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the PPD prediction devices, systems, and methods. Certain features may be added, removed, or modified without departing from the spirit of the claimed subject matter.

In an example, a machine learning algorithm is trained using EHR data obtained during a population survey. In an example study, EHRs were used from Weill Cornell Medicine and New York-Presbyterian Hospital from 2015 to 2017 as the data source. The study data are represented using Observational Medical Outcomes Partnership (OMOP) common data model and include patient socio-demographics, timestamped outpatient and inpatient diagnoses, and timestamped medication prescription.

Pregnant women with fully completed antenatal care procedure at the hospital and with a birth of a singleton infant were included in the study. The exclusion criteria were: (1) whose with unknown length of gestational weeks, (2) those with missing information from at least one trimester during pregnancy.

Clinical assessment of PPD was used as the outcome in this example. The main outcome was defined based on the Statistics Canada and International Classification of Diseases, 10th Revision (ICD-10-CM) codes O99.3 and O99.34 codes as well as their ICD-9-CM equivalents for a diagnosis of PPD within 12 months after childbirth. Patients' birthdate, race, maternal status, average body mass index (BMI), gestational week, and delivery type as time-independent predictors, and medication prescriptions and diagnoses were considered at each clinical visit as the time-dependent predictors.

Age was calculated as baseline age at the first visit of prenatal care. Marital status was extracted from unstructured clinical notes and categorized as single (unmarried, divorced and windowed), married, and unknown. Race groups included White, Asian, American Indian or Alaska Nation, Black or African American, Other combinations not described, and Unknown. Native Hawaiian, Other Pacific Islander, and other racial combinations were included as “Other combinations not described.” Gestational weeks were computed using the delivery dates and gestational checkup weeks. The specific trimester of medication prescription and diagnoses were identified by the time interval between each event and delivery. Trimester of pregnancy is defined as follows: first trimester (0-12 weeks), second trimester (13-28 weeks), and third trimester (29 weeks—gestation). All diagnoses were represented as Systematized Nomenclature of Medicine-Clinical Terms (SNOMED-CT) codes. Medication and dosage were standardized by Anatomical Therapeutic Chemical (ATC) Classification System.

In order to perform variable selection for the prediction model, the variables were first selected above the median frequency for all variables. Then, univariate logistic regression (LR) analyses were performed, in which factors with p-values below 0.05 were assigned as potential predictive factors.

In an example, six machine learning models, including Support Vector Machine (SVM), Random Forest (RF), Naïve Bayes, L2-regularized LR, Extreme Gradient Boosting (XGBoost) and Decision Tree were used to build PPD prediction models. Each model's performance was evaluated using the area under the receiver-operator curve (AUC) in 10-fold cross validation. All machine learning and statistical analyses were performed with R version 3.4.3 (R Foundation for Statistical Computing, Vienna, Austria).

SVM is a classifier which transforms input data into a multi-dimensional hyperplane using kernels to discriminate two classes. RF is an ensemble learning method that operates by constructing a multitude of decision trees and outputting the class that is voted by a majority of the trees. Naïve Bayes classifier uses the Bayes Theorem to predict member-ship probabilities for each class by assigning class with the highest probability as the most likely class. LR is a regression model with a binary dependent variable. L2-regularized LR tunes and generalizes the model in order to balance the bias-variance trade off. XGBoost is a scalable tree boosting algorithm which trains a sequence of models to minimize errors made by existing models. Lastly, decision Tree predicts class membership by inferring decision rules from the training data. For all models, an over-sampling method was applied to the training data as the outcome was imbalanced (see Table 1). Oversampling is a popular meth-od in dealing with class imbalance problems, which changes the training sets by repeating instances in the minority training set.

In order to provide more interpretability to the model, the association of predictors was examined categorically with PPD. Models with were combined different feature compositions (see Table 3). First, the temporality of the features was examined by grouping medication prescribed by trimesters. Then, feature categories were examined by building models with socio-demographic information only, medication information only, diagnostic information only, and medication combined diagnostic information. Lastly, a model was built with only variables selected using the univariate LR. The Pearson correlation of variables was tested to prevent multicollinearity. If the correlation coefficients were greater than 0.8, variables were combined. Only variables whose associations with PPD were statistically significant were selected. Odds ratios with 95% confidence intervals (CI) and p-values are presented in Table 4.

Table 1 shows the characteristics of pregnant women with and without PPD. Results are presented as the mean±standard deviation for continuous variables and N (%) for categorical variables. A p-value less than 0.05 is considered statistically significant in statistical analyses. Among the studied population, 9,980 episodes of pregnancy were identified. There was a significant difference in age between two groups using a student t-test. The mean age was 33.92 (SD 4.51) years old in non-PPD group and was 34.36 (SD 4.61) years old in the PPD group. The pre-pregnancy average BMI in PPD group is higher than that in non-PPD group. There were significant differences in race between the PPD and non-PPD groups using a Fisher exact test. The number of single mothers is higher in the PPD group than non-PPD group (23.15% vs. 15.96%).

TABLE 1
Baseline characteristics of pregnant women.

| Variables | Non-PPD | PPD |
|---|---|---|
| N | 9211 | 769 |
| Age, years* | 33.92 ± 4.51 | 34.36 ± 4.61 |
| Pre-pregnancy BMI, kg/m²* | 23.61 ± 4.41 | 23.93 ± 4.99 |
| Race* | | |
| White | 4801(52.12) | 478(62.16) |
| Asian | 1455(15.80) | 62(8.06) |
| American Indian or Alaska Nation | 30(0.33) | 3(0.39) |
| Black or African American | 492(5.34) | 45(5.85) |
| Other combinations not described | 1067(11.58) | 90(11.70) |
| Not known | 1366(14.83) | 93(12.09) |
| Marital Status* | | |
| Single | 1470(15.96) | 178(23.15) |
| Married | 4610(50.05) | 416(54.10) |
| Not known | 3131(33.99) | 175(22.76) |
| Cesarean section* | | |
| No | 8352(90.67) | 679(88.29) |
| Yes | 859(9.33) | 90(11.70) |

*Significant statistical difference found between two groups.

TABLE 2
Prediction Results.

| Machine learning technique | AUC | Sensitivity | Specificity |
|---|---|---|---|
| SVM | 0.79 | 0.894 | 0.580 |
| L2 LR | 0.78 | 0.887 | 0.594 |
| RF | 0.78 | 0.959 | 0.391 |
| Naïve Bayes | 0.78 | 0.867 | 0.616 |
| XGBoost | 0.77 | 0.915 | 0.527 |
| Decision Tree | 0.69 | 0.986 | 0.386 |

Prior to variable selection, 256 variables were extracted including socio-demographic characteristics, disease diagnoses, and medications across 3 trimesters. Then 98 potential predictors were identified using univariate LR analyses. Among the selected variables, 71 variables were diagnoses and 22 were medications. Results from the 6 machine learning models using all 98 predictors are shown in Table 2. AUC for different classifier was the highest with SVM (0.79), followed by L2-regularized LR (0.78), RF (0.78), Naïve Bayes (0.78), XGBoost (0.77), and was the lowest of 0.69 for the Decision Tree. The sensitivity of different models were further computed. In the model, Decision Tree had the highest sensitivity (98.6%), followed by the RF (95.9%), XGBoost (91.5%), SVM (89.4%), LR (88.7%) and naïve Bayes (86.7%). The specificity was highest for naïve Bayes (61.6%), followed by L2-regularized LR (59.4%), SVM (58.0%), XGBoost (52.7%) and Decision Tree (38.6%).

Using SVM, the best performing model, model performance was investigated using different feature compositions, presented in Table 3. The AUC for the model using only first, second and third trimester information was 0.66, 0.64, and 0.65, respectively. The AUC for the model with variables in both first trimester and second trimester, second trimester and third trimester was 0.69 and 0.72, respectively, both of which lower than the complete feature set. In addition, the AUC for the model with only demographic variables was 0.60. The AUCs for the diagnoses model or medication classes model were 0.72 and 0.65, respectively. When medications and diagnoses were combined together, the AUC increased to 0.76.

TABLE 3
Prediction Results in different variable combinations.

| Predictors | AUC | Specificity | Sensitivity |
|---|---|---|---|
| Trimesters | | | |
| 1^{st} | 0.66 | 0.855 | 0.428 |
| 2^{nd} | 0.64 | 0.831 | 0.424 |
| 3^{rd} | 0.65 | 0.867 | 0.424 |
| 1^{st} + 2^{nd} | 0.69 | 0.908 | 0.307 |
| 2^{nd} + 3^{rd} | 0.72 | 0.854 | 0.524 |
| Categories | | | |
| Demographic | 0.60 | 0.551 | 0.609 |
| Diagnose | 0.72 | 0.850 | 0.560 |
| Medication | 0.65 | 0.882 | 0.389 |
| Diagnose + Medication | 0.76 | 0.875 | 0.577 |
| Logistic-selected | 0.76 | 0.892 | 0.588 |

The univariate LR identified 26 predictors out of the 98 predictors whose associations with PPD have significant and meaningful odds ratios for PPD (Table 4). The AUC using 26 important features were lower than using the whole features. None of the reduced models had performance as well as the full model (Table 3). In Table 4, amongst factors related to diagnoses during pregnancy, obesity, anxiety, depressive disorder, mental disorder, threatened miscarriage in first trimester; abnormal weight gain, anxiety, depressive disorder, diarrhea, mental disorder, premature labor, muscle pain, vomiting in second trimester; and anxiety, abdominal pain, backache, hypertensive disorder, mental disorder, palpitations, major depression, single episode in third trimester were found to be associated with increased odds of PPD. Among medications, the use of antidepressants during pregnancy, and anti-inflammatory agents in second trimester were associated with increased odds of PPD. Among the predictors, hormone use had no associations with PPD, despite their mention in previous literature.

TABLE 4
Association between Predictors and PPD.

| Variables | | OR(95% CI) | P |
|---|---|---|---|
| Marital status | | | |
| Single | | REF | |
| Married | | 0.82(0.65, 1.04) | 0.096 |
| Not known | | 0.58(0.45, 0.76) | <0.001 |
| Race | | | |
| White | | REF | |
| Asian | | 0.54(0.39, 0.74) | <0.001 |
| American Indian or Alaska Nation | | 0.54(0.07, 2.34) | 0.475 |
| Black or African American | | 0.68(0.43, 1.04) | 0.084 |
| Other combinations not describe | | 0.82(0.62, 1.07) | 0.158 |
| Declined | | 0.95(0.71, 1.25) | 0.713 |
| Diagnose | | | |
| Anxiety | 1^{st} | 10.49(6.22, 17.75) | <0.001 |
| Depressive disorder | 1^{st} | 18.58(9.73, 35.93) | <0.001 |
| Mental disorder | 1^{st} | 4.04(1.34, 12.02) | 0.013 |
| Obesity | 1^{st} | 1.75(1.03, 2.85) | 0.031 |
| Threatened miscarriage | 1^{st} | 1.72(1.15, 2.51) | 0.007 |
| Abnormal weight gain | 2^{nd} | 2.84(1.22, 5.98) | 0.010 |
| Anxiety | 2^{nd} | 4.08(2.27, 7.28) | <0.001 |
| Depressive disorder | 2^{nd} | 4.35(1.30, 15.71) | 0.021 |
| Diarrhea | 2^{nd} | 2.78(1.28, 5.54) | 0.006 |
| Mental disorder | 2^{nd} | 6.87(2.36, 20.41) | <0.001 |
| Premature labor | 2^{nd} | 5.20(1.92, 12.75) | 0.001 |
| Muscle pain | 2^{nd} | 3.74(1.35, 9.05) | 0.006 |
| Vomiting of pregnancy | 2^{nd} | 2.43(1.02, 5.47) | 0.037 |
| Anxiety | 3^{rd} | 9.52(5.67, 16.00) | <0.001 |
| Abdominal pain | 3^{rd} | 1.71(1.08, 2.62) | 0.018 |
| Backache | 3^{rd} | 3.68(1.67, 7.41) | 0.001 |
| Hypertensive disorder | 3^{rd} | 3.24(1.49, 6.76) | 0.002 |
| Mental disorder | 3^{rd} | 2.84(1.29, 6.16) | 0.009 |
| Major depression, single episode | 3^{rd} | 5.49(2.30, 12.78) | <0.001 |
| Palpitations | 3^{rd} | 2.38(1.010, 5.022) | 0.033 |
| Medication | | | |
| Antidepressants | 1^{st} | 13.47(7.58, 24.13) | <0.001 |
| Antidepressants | 2^{nd} | 10.84(4.86, 25.08) | <0.001 |
| Antidepressants | 3^{rd} | 24.21(12.39, 49.20) | <0.001 |
| Anti-inflammatory agents | 2^{nd} | 16.64(1.73, 158.16) | 0.009 |

In an example, six machine learning models were employed to predict PPD using EHR data. Experimental results demonstrated the feasibility of the approach for PPD risk prediction based on information available during prenatal care in EHR. Several disease diagnoses and medications were found during pregnancy that potentially contribute to the prediction of PPD.

The performances of the model using variables in one specific trimester only, both first and second trimesters or both in second and third trimesters were not as good as using variables in whole prenatal care. Thus, the findings potentially suggest that screening should consider health and health service utilization throughout the pregnancy period. When the variables were separated into demographic variables, diagnoses variables, and medication variables, disease diagnoses had the best performance in predicting PPD. Although, using the combination of disease diagnoses with medication improved the performance in predicting PPD than using disease diagnoses alone. Again, more comprehensive information provides more improved prediction performance.

The data set included multiple features in EHR data. In some examples, SVM had the best performance. However, the difference across the performance of SVM, L2-regularized LR, RF, Naïve Bayes, and XGBoost was minimal, although differences existed with respect to sensitivity and specificity. Other machine learning models may be used instead or in addition. The AUC of the decision tree model was the lowest compared with the other five models. This may be explained by the tendency of the decision tree to depend on single variables in generating decision rules.

Several associations may be consistently predictive. These include race as demographics and threatened abortion, prenatal mental disorder, in particular, depression disorder, anxiety, and single episode major depression, backache in the third trimester, and muscle pain in second trimester, as diagnoses. Pain is often expressed as symptoms of depressive disorder. Thus, the results indicate that there is a strong need for perinatal interventions to focus on expectant mothers' mental health as prevention for PPD.

Correspondingly, antidepressant use across three trimesters may in some cases be a strong predictor for developing PPD in the future. The treatment of women with depression or other psychiatric diseases during pregnancy or postpartum is a complex clinical challenge. In some examples, women who had antidepressant treatment during pregnancy were less likely to report postnatal depressive symptoms, compared with the nonmedicated counterpart. However, discontinued antidepressant medications during pregnancy was a risk factor for PPD. On the other hand, antidepressants use during pregnancy might be associated with gestational hypertension and preeclampsia. Analgesic was not found as an independent risk factor for PPD, although its association was reported in previous literature.

Some embodiments include certain limitations in the current work. First, since medications were combined by ATC classes, drug use was unable to be differentiated by specific dosage levels. In other embodiments using larger data set, different sources of disease diagnoses can be identified and combined, and the dose-response relationship with medications and PPD can be considered. Second, the machine learning methods used in some embodiments may be standard methods, and the oversampling method used to handle the imbalance data may have contributed to overfitting and impacted model performance. More advanced machine learning methods such as the neural network models will be used to improve AUC in future work. Third, since EHRs were used from a single health system, the data may miss mothers diagnosed with PPD outside of the health system, as well as information on those who were seen by clinicians outside of the health system before their pregnancy. Future studies will try to leverage multi-site dataset to minimize missing and erroneous data points. Lastly, the aim of some embodiments is to predict PPD rather than evaluating the causal relationships between variables in the pregnancy period and PPD. Future studies will use causal inference methods to control for potential and time-dependent confounding.

In some embodiments, promising PPD prediction results were demonstrated using a machine learning approach with information on patient demographics, diagnoses, and medications available from EHRs. The goal is to create an accurate PPD prediction model to identify risk factors for PPD and facilitate effective screening of mothers who may require early intervention for PPD using EHR. It is envisioned that the model may be integrated with the EHR system for provider-facing CDS or with a mobile or web platform to be used as patient-facing CDS in other embodiments.

Clinically, a history of mental illness is the most significant risk factor (Meltzer-Brody et al., 2018; Stewart and Vigod, 2016). Social determinants of health (SDoH), including poor marital relationship, low socioeconomic status, and stressful life events can also be known contributors to increased PPD risk (Biaggi et al., 2016). New research indicates that there may be additional biomarkers associated with the risk for developing PPD such as excessive proinflammatory immune system activation, possible disruptions in fatty acid metabolism, disruptions in hypothalamic-pituitary-adrenal (HPA) functioning, altered neurosteroid physiology, and genetic and epigenetic signatures (Serati et al., 2016). The importance of PPD prevention and timely intervention cannot be overstated. The American College of Obstetricians and Gynecologists (ACOG) (Committee, 2018), the American Academy of Pediatrics (AAP) (Earls et al., 2019), the US Preventive Services Task Force (O'Connor et al., 2019), and several other organizations (Stewart and Vigod, 2016) have guidelines and recommendations for universal PPD screening as part of usual care during pregnancy and the postpartum period. Current PPD prevention strategies focus on secondary rather than primary prevention, using questionnaire-based screening instruments such as the Edinburgh Postnatal Depression Scale (EPDS) (Cox et al., 1987) and Patient Health Questionnaire-9 (PHQ-9) (Löwe et al., 2004) to detect symptoms. Primary prevention techniques intervene in an illness course prior to symptom onset while secondary prevention techniques intervene soon after the symptom onset, but prior to the full manifestation of the illness. Unfortunately, it has been demonstrated that in women known to be at high risk of PPD, delaying intervention until the onset of symptoms only mildly attenuates risk for depression, while intervening with appropriately targeted prevention before the onset of symptoms substantially mitigates depression relapse risk (Cohen et al., 2006). In addition to being “too little, too late” from a clinical perspective, these screening tools present major feasibility problems for both large and smaller health systems (Beck and Gable, 2000; Gjerdingen and Yawn, 2007). In order to come into compliance with current screening recommendations, obstetric practices often require substantial change, including not just changes to clinical workflows, but also staffing changes, new electronic health records (EHR) workflow builds, collaboration with referral networks, and investment in staff and provider training. Even then, further challenges persist such as mental health-related stigma, limitations in provider time, attention, and expertise, and scarcity in specialized mental health treatment resources.

Taking a primary prevention approach has the promise of reducing the investment and resources required to address PPD while at the same time reducing the incidence of PPD rates. In this work, to identify signals that may suggest elevated future risk of PPD, a primary prevention approach is employed that is data-driven, leveraging machine learning applications to EHR data (Loudon et al., 2016; Wang et al., 2019). EHR data can be collected and analyzed routinely on a large scale using machine learning, as demonstrated by successful data-driven clinical decision support (Shortliffe and Sepnlveda, 2018) applications that assist with decision making across clinical conditions (Goldstein et al., 2017; Liang et al., 2019; Rajkomar et al., 2018; Tomasev et al., 2019). The framework was implemented and evaluated using data available at different time intervals during pregnancy (12-week, 18-week, 24-week, and 30-week) during pregnancy and after childbirth.

The data-driven primary intervention approach provides an opportunity for individualized therapeutic interventions such as changing screening timelines, engaging with appropriate preventive strategies, or tailoring clinician PPD counseling time according to a patient need. Some embodiments develop an EHR-based machine learning framework for identifying women at risk for PPD (Jiménez-Serrano et al., 2015; Tortajada et al., 2009; Wang et al., 2019; Zhang et al., 2020).

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

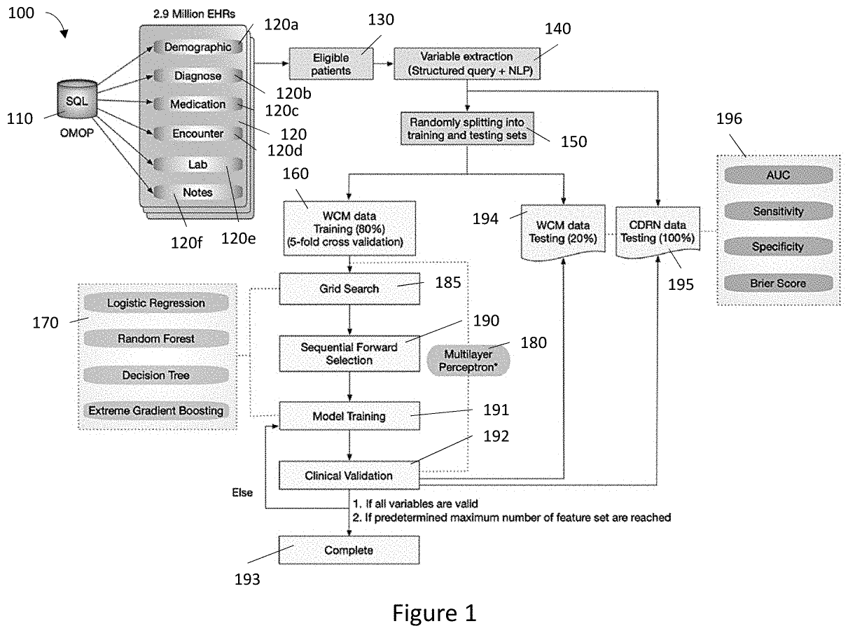

FIG. 1 shows the schematic diagram 100 of an example PPD prediction framework that describes the various steps involved in data preprocessing and risk model development, in accordance with at least one embodiment of the present disclosure. The schematic diagram 100 includes a database 110 including electronic health records (EHR) for a plurality of patients. The EHR may for example include demographic data 120a, diagnosis data 120b, prescription data 120c, encounter data 120d (e.g., emergency department visits), lab data 120e (e.g., weight), and notes 120f (e.g., marital status). The schematic diagram 100 also includes a list of selected patients 130, a variable extraction step 140, a splitting step to split the eligible patients into a training dataset 160 and a testing or validation dataset 194. The testing data can be used to extract quality metrics 196, which may for example include area under the curve, sensitivity, specificity, and Brier score. The schematic diagram 100 also includes a machine learning classifier 170 comprising at least one of a logical regression, random forest, decision tree, or extreme gradient boosting classifier. The schematic diagram 100 also includes a multilayer perceptron 180 that performs a grid search 185, sequential forward selection 190, model training step 191, a clinical validation step 192 on the testing data 195, and an ending step 193.

The machine learning model training can be optimized using sequential forward selection (SFS) (T. and G., 2015)—a greedy search algorithm that searches for the combination of features that returns the maximum algorithm discriminatory power (Bradley, 1997). Starting with an empty feature set, SFS iteratively examines each feature combination such that the algorithm's performance can be maximized until the stopping criteria for the search is reached (FIG. A1) (T. and G., 2015). Five machine learning algorithms were trained, including random forest, decision tree, extreme gradient boosting (XGboost), regularized logistic regression, and multilayer perceptron (MLP). These algorithms were developed by iteratively splitting the data available to detect collective patterns across features in the subset of the data that maximally discriminate outcome classes, followed by testing the performance on the held-out data. This training process allowed us to develop prediction algorithms that are generalizable to unseen data.

Algorithm parameters were determined using a grid search for each algorithm that comprehensively searched for the best hyperparameters and parameters that resulted in the highest model performance as measured by area under the receiver operating characteristic curve (AUC). The stopping criteria for SFS were defined as 1) no increase in the AUC by 0.001 after 10 consecutive iterations, or 2) the predetermined maximum number of feature set has been reached. SFS was performed separately for women with, and without, mental health history to ensure that the model can predict for both types of patients when in actual use. Features may be selected from both SFS into a single feature set such that a single algorithm can be used for patients with and without a history of mental illness. Using the combined features, each of the machine learning algorithms can be trained using 5-fold cross-validation.

An end-to-end framework extracts features from EHR data for processing, including demographics, clinical diagnoses, medication prescriptions, laboratory results, and unstructured clinical notes. These data can be sent to an optimization process to select important features and incorporated in multiple machine learning algorithms including regularized logistic regression, random forest, decision tree, extreme gradient boosting (XGboost), and multilayer perceptron (MLP) (Bishop, 2006) to predict the risk of PPD. The framework was implemented and evaluated using data available at different time intervals during pregnancy (12-week, 18-week, 24-week, and 30-week) during pregnancy and after childbirth.

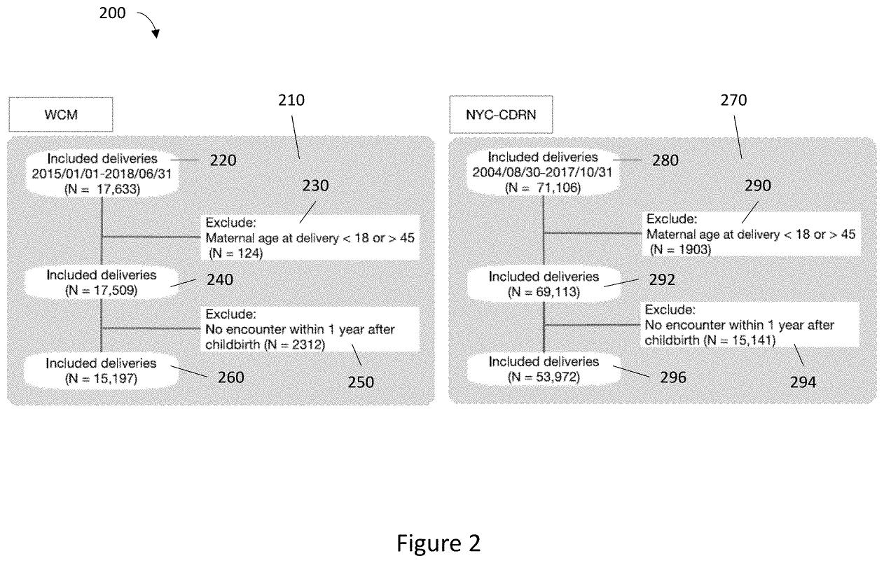

FIG. 2 shows the inclusion and exclusion criteria 200 of an example study, in accordance with at least one embodiment of the present disclosure. The inclusion and exclusion criteria 200 include the Weill Cornell Medicine (WCM) training data 210 and the New York City Clinical Data Research Network data (NYC-CDRN) validation data 270. The WCM data 210 includes total deliveries 220, deliveries excluded for mother's age 230, deliveries included for mother's age 240, deliveries excluded for no health encounters 250, and deliveries included for health encounters 260. The NYC-CDRN data 270 includes total deliveries 280, deliveries excluded for mother's age 290, deliveries included for mother's age 292, deliveries excluded for no health encounters 294, and deliveries included for health encounters 296.

From the EHR available for the study, all pregnant women with fully completed antenatal care procedures who had live births of infants were included in the study. The exclusion criteria included (1) maternal age below 18 or above 45, or (2) lack of outpatient, inpatient or emergency room encounter information in the EHR data within 1 year following childbirth. Participants with a prior history of mental illness and participants with active mental illness were not excluded to ensure clinical applicability in real implementation (FIG. 2). The study was approved by the Institutional Review Board at Weill Cornell Medicine (IRB protocol #1711018789). Data extraction and analysis were performed in 2019.

A total of 15,197 deliveries from January 2015 to June 2018 were included in the analysis, excluding 124 women below age 18 or above age 45 at the time of delivery, and 2,312 women without records of clinical encounters within 1 year following childbirth (FIG. 2). Study data were randomly split into training (N=12157) and testing (N=3040) using cross-validation. The validation set contained 53,972 deliveries from August 2004 to October 2017, after excluding 1,903 deliveries by women below age 18 or above age 45 and 15,141 deliveries without encounters recorded within 1 year after childbirth (FIG. 2). The prevalence of depression was 6.7% (N=1,010) and 6.5% (N=3,513) in the WCM and NYC-CDRN datasets, respectively.

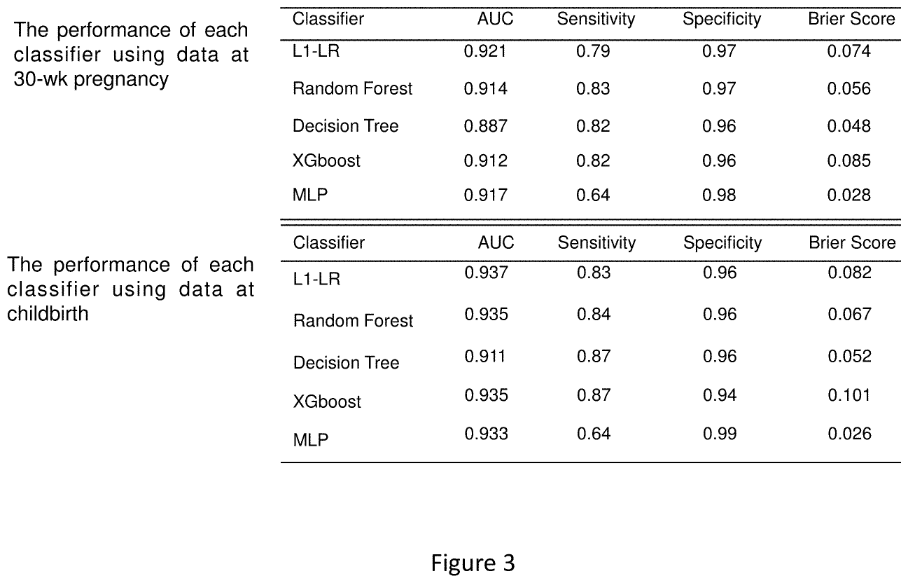

FIG. 3 shows model performance for the WCM training data, in accordance with at least one embodiment of the present disclosure. For algorithm development, EHR data including demographics, diagnoses, medication prescriptions, procedures, laboratory measurements, and social determinants of health (SDoH) including the built environment characteristics such as distance to public transportation and green space on eligible patients were obtained at Weill Cornell Medicine (WCM) and NewYork-Presbyterian Hospital in New York City, USA between January 2015 and June 2018. For algorithm validation, EHR data was derived from multiple health systems across New York City affiliated to the Patient-Centered Outcomes Research Institute funded New York City Clinical Data Research Network data (NYCCDRN) between August 2004 and October 2017 (Kaushal et al., 2014). One example randomly selected 80% of the data from WCM as the training set including cross-validation and model tuning, and held the remaining 20% as the test set individually. The NYC-CDRN data was used solely as a validation set.

Both datasets were represented using Observational Medical Outcomes Partnership (OMOP) Common Data Model to record patient demographics, encounter records, diagnostic codes, procedures, prescription medications, and laboratory measurements (Overhage et al., 2012). Diagnoses, laboratory measurements, and procedures are represented as SNOMED codes, Logical Observation Identifiers Names and Codes (LOINC), and Current Procedural Terminology (CPT) codes, respectively. Medications were standardized using the ATC classification system. In addition, marital status was extracted from unstructured clinical notes using regular expression-based searches, and individuals were classified as married or not married (single/divorced/widowed) at the time of childbirth. Age was calculated as the time difference between childbirth and delivery dates. Mental health history before pregnancy was defined as having at least one diagnosis including organic disorders, substance-related disorders, schizophrenic/psychotic disorders, mood disorders, anxiety disorders, personality disorders, and other psychiatric disorders (Canada, 2015). Features with frequencies below 10 may be omitted to remove rare events during pregnancy. Mean values were used to perform the imputation of missing numerical values. Discrete features, such as clinical diagnoses, prescribed medication, were coded as dummy features (Rodriguez et al., 2018). Numeric features were normalized in the scale of −1 to 1. Statistical comparison across the PPD and non-PPD group was performed using Stata 14. Independent sample T-test assuming unequal variances and Chi-Square test was used for continuous and categorical variables as appropriate.

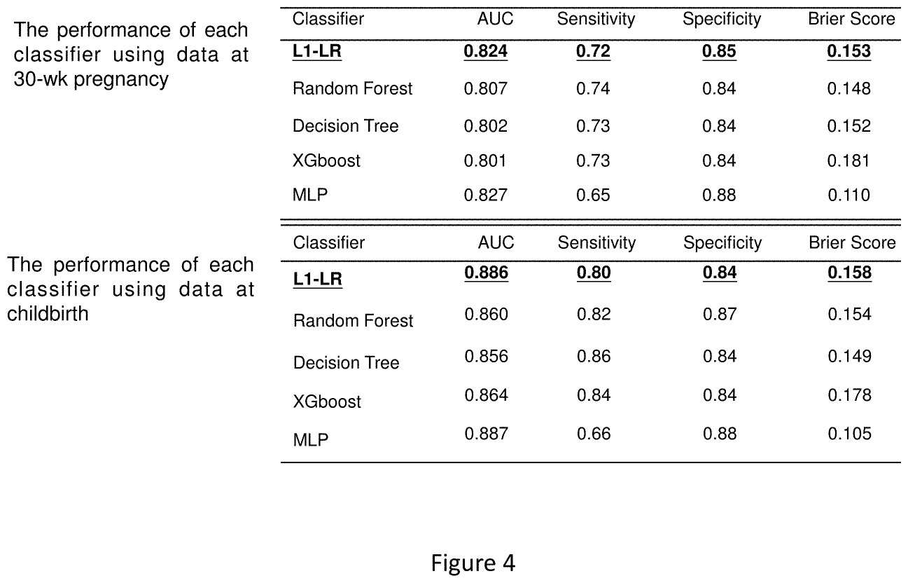

FIG. 4 shows model performance for the NYC-CDRN/INSIGHT validation data, in accordance with at least one embodiment of the present disclosure. The best-performing model is the logistics regression classified trained using clinical features related to mental health history, medical comorbidity, obstetric complications, medication prescription orders, and patient demographic characteristics. The model performances as measured by area under the receiver operating characteristic curve (AUC) are 0.937 (95% CI 0.912-0.962) and 0.886 (95% CI 0.879-0.893) in the development and validation datasets, respectively. The model performances were consistent when tested using data ending at multiple time periods during pregnancy and at childbirth. The prevalence of PPD represents a treatment prevalence and is likely lower than the illness prevalence. EHRs and machine learning offer the ability to identify women at risk for PPD early in their pregnancy. This may facilitate scalable and timely prevention and intervention, reducing negative outcomes and the associated burden.

The outcome is defined as having a diagnosis of PPD within 1 year of childbirth. A PPD diagnosis was defined using Systematized Nomenclature of Medicine (SNOMED) codes and the use of antidepressants within 1 year following childbirth (Dietz et al., 2007; Stewart and Vigod, 2016). The specific SNOMED codes for PPD definition are listed in Appendix (Table A1). The use of antidepressants was defined by Anatomical Therapeutic Chemical (ATC) codes under N06A (Petersen et al., 2018). To ensure that antidepressants were primarily used for treatment of mental health conditions, and not for other indications such as pain, an example study further excluded the following medications: Amitriptyline, Clomipramine, Duloxetine, Flupentixol, and Nortriptyline (Schofield et al., 2016).

A total of 15,197 deliveries from January 2015 to June 2018 were included in the analysis, excluding 124 women below age 18 or above age 45 at the time of delivery, and 2,312 women without records of clinical encounters within 1 year following childbirth. Study data were randomly split into training (N=12157) and testing (N=3040) using cross-validation. The validation set contained 53,972 deliveries from August 2004 to October 2017, after excluding 1,903 deliveries by women below age 18 or above age 45 and 15,141 deliveries without encounters recorded within 1 year after childbirth (FIG. 2). The prevalence of depression was 6.7% (N=1,010) and 6.5% (N=3,513) in the WCM and NYC-CDRN datasets, respectively.

We found significant differences in age, the number of emergency department (ED) visits, and racial distribution between PPD and non-PPD groups in the training and validation data, respectively. The average age at the time of delivery was 33.68 (SD=4.54) in the non-PPD group and 34.56 (SD=4.39) in the PPD group of patients in the WCM dataset (p-value<0.001), and 28.87 (SD=6.20) and 30.70 (SD=6.13) in the CDRN dataset, respectively (p-value<0.001). The number of emergency room visits in the PPD group was higher than the non-PPD group in both the WCM (1.68±1.55 vs. 1.32±1.24, p-value<0.001) and NYC-CDRN (6.30±9.97 vs. 5.37±6.87, p-value<0.001) datasets. The training and validation datasets had different distribution of PPD across racial groups. In the WCM data, the incidence rate of PPD was the highest among White women (8.8%) and the lowest among Asian women (3.0%). In the CDRN data, the rate of PPD was the highest among White women (12.43%), Black patients had the lowest rates (4.76%).

Using SFS, 32 features were selected to be incorporated in the algorithm related to patient demographic statuses, health service utilization, mental health history, newly diagnosed mental health conditions during pregnancy, other obstetric and/or medical diagnoses during pregnancy, and vital signs. As shown in Table 2, the majority (28 out of 32) of the features included in the algorithm have statistically significant association with the outcome. Features that are indicative of past and current mental health conditions and being single mothers were associated with higher odds of a PPD diagnosis. Additionally, complications during pregnancy such as palpitations, diarrhea, vomiting, and abdominal pain also were associated with higher odds of a PPD diagnosis. Health service utilization including medication prescriptions such as Beta blocking agents, delivery by cesarean, and emergency department (ED) visits were also associated with higher odds of a PPD diagnosis. Having an Asian race was associated with lower odds of a PPD diagnosis. FIG. A2 in the Appendix shows the Pearson correlation among the features.

Logistic regression with L2 regularization was found to be the best performing algorithm using data available up to childbirth. The AUC was 0.937 (95% CI: 0.912-0.962) and 0.886 (95% CI: 0.879-0.893) in the WCM and NYC-CDRN datasets, respectively. The AUC was lower in the validation dataset potentially due to the lack of certain features such as marital status which was available only in the WCM dataset. While evaluating algorithms at different periods during the pregnancy, one can observe a steady performance with respect to AUC of 0.921, 0.919, 0.922, 0.921, and 0.937 using data extracted up to 12 weeks, 18 weeks, 24 weeks, 30 weeks of gestation, and at childbirth, respectively. The steady performance may be explained by the early availability and invariability of the predictive features. In the NYC-CDRN (previously known as INSIGHT) dataset, one can observe an increase in algorithm performance as more data accumulate over time, with an AUC of 0.810, 0.817, 0.821, 0.824, and 0.886 at 12 weeks, 18 weeks, 24 weeks, 30 weeks of gestation, and lastly at childbirth, respectively. While negative predictive values may be close to 1 for nearly all models across time periods, positive predictive values can be low, especially in the validation site. This could be explained by the relative low prevalence of PPD and the high frequency of the patients who were not diagnosed to have PPD (based on the identified criteria), but were predicted so.

False-positive and false-negative results from the algorithm were evaluated by manual chart reviews of a randomly selected 150 cases that were incorrectly classified by the logistic regression classifier. The cases had 140 and 10 false positives and false negatives, respectively. PPD diagnosis after the study period and lack of proper coding were identified as two potential reasons for the false positives and negatives. For example, the manual chart review identified that 45% of the patients incorrectly predicted to develop PPD by the prediction algorithm were in fact women who were noted to be suffering from PPD in the clinical notes. Furthermore, 34% of the PPD mentions in the notes were made one year after childbirth, beyond our study period. Thus, the incorrect predictions were due to the lack of good coding practices for PPD, a phenomenon that is frequently observed in other observational mental health studies using EHRs (Stewart et al., 2019). The availability of predictors related to mental health history also presented challenges. For example, the error analysis identified the history of anxiety and depression on 36.4% of false negative cases through manual chart review. For these patients the mental health history was not coded in the structured EHR data. Extraction of features using natural language processing techniques may facilitate higher performance in some embodiments.

All pregnant women may be at risk for postpartum depression. In some cases, strong predictors of postpartum depression may include mental health history (Forman et al., 2000), thyroid dysfunction and hypertensive disorders (Le Donne et al., 2017; Strapasson et al., 2018), heart palpitations (Alijaniha et al., 2016; Barsky et al., 1994), use of Beta blockers and antihistamines (Yudofsky, 1992, Gerstman et al., 1996; Ozdemir et al., 2014), previous C-sections (Carter et al., 2006; Xu et al., 2017), and number of ED visits (Sheen et al., 2019). In some cases, family history contributes 40% to the risk of developing PPD. Other risk factors may include domestic violence, lack of familial support, poor income and education level, biochemical, immunological and endocrinological risk factors, and other factors.

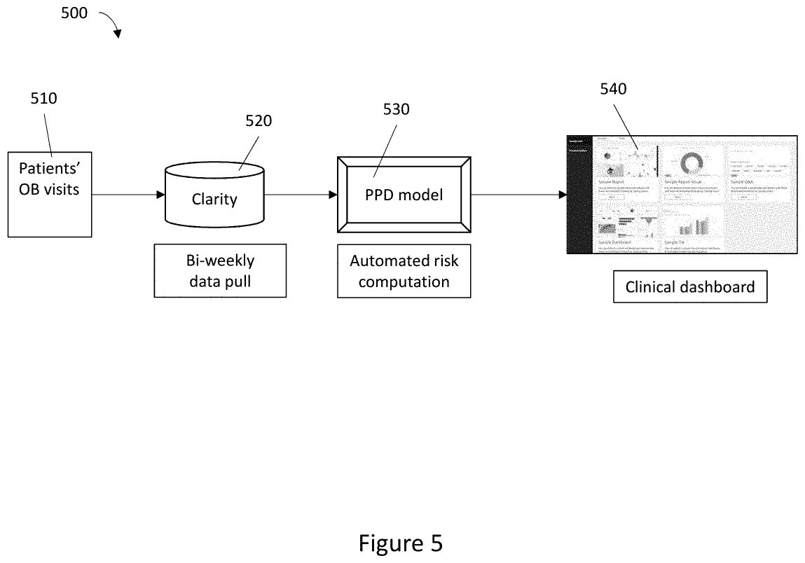

FIG. 5 shows a schematic in block diagram form of an example clinician workflow 500, in accordance with at least one embodiment of the present disclosure. The clinician workflow 500 includes a patient visit 510 to one or more obstetric-gynecologic (OB-GYN) practitioners, a bi-weekly data pull 520, risk modeling 530 for the probability of the patient developing post-partum depression, and a clinical dashboard 540. The data pull 520 may for example involve patient-reported symptom tracking (e.g., with an application running on a mobile device), wearables integration for symptom tracking, an EHR-integrated patient-provider communication portal, an EHR-integrated telemedicine platform, Asynchronous Cognitive Behavior Therapy (CBT) Intervention, and/or a peer network. In addition to EHR, the data pull may include social determinants of health (SDOH) information by, for example, linking EHR with 11-digit census tracts (e.g., from a government census database) to obtain or deduce patient demographic traits such as income, social stresses, residence location, citizenship, insurance, race, religion, marital status or relationship status, employment, access to transportation, and Internet access. An example list of weighted risk factors is presented in Table 5.

TABLE 5
Weighted PPD risk factors

| Variables | OR (95% CI) | P | modifiable |
|---|---|---|---|
| Anxiety history | 108.90(87.55, 135.44) | <0.001 | N |
| Antidepressants | 94.14(59.52, 148.89) | <0.001 | N |
| Mood disorder history | 43.80(34.75, 55.21) | <0.001 | N |
| Depression in pregnancy | 27.97(20.22, 38.68) | <0.001 | N |
| Anxiety in pregnancy | 27.93(21.05, 37.07) | <0.001 | N |
| Mental disorder in | 22.27(15.99, 31.01) | <0.001 | N |
| pregnancy | | | |
| Other disorder history | 18.22(14.46, 22.96) | <0.001 | N |
| Palpitations | 2.65(1.75, 3.99) | <0.001 | N |
| Diarrhea | 2.54(1.70, 3.79) | <0.001 | N |
| Vomiting in pregnancy | 2.52(1.86, 3.40) | <0.001 | N |
| Hypertensive disorder | 2.25(1.34, 3.77) | 0.002 | N |
| Acute pharyngitis | 2.19(1.30, 3.66) | 0.003 | N |
| Hemorrhage in early | 2.09(1.18, 3.68) | 0.011 | N |
| pregnancy antepartum | | | |
| White | 2.03(1.75, 2.35) | <0.001 | N |
| Threatened miscarriage | 1.99(1.52, 2.59) | <0.001 | N |
| Abdominal pain | 1.98(1.58, 2.48) | <0.001 | N |
| Migraine | 1.98(1.02, 3.86) | 0.044 | Y |
| Beta blocking agents | 1.91(1.24, 2.95) | 0.003 | Y |
| Antihistamines for | 1.81(1.43, 2.30) | <0.001 | N |
| systemic use | | | |
| Hypothyroidism | 1.64(1.34, 2.01) | <0.001 | N |
| Placental infarct | 1.62(1.11, 2.37) | 0.013 | N |
| Single (vs. Married) | 1.59(1.33, 1.91) | <0.001 | N |
| Deliveries by cesarean | 1.47(1.25, 1.72) | <0.001 | Y |
| Direct acting antivirals | 1.44(1.06, 1.94) | 0.018 | Y |
| Primigravida | 1.39(1.19, 1.61) | <0.001 | N |
| Pre-eclampsia | 1.38(0.67, 2.87) | 0.386 | N |
| Other antibacterials | 1.33(0.97, 1.80) | 0.073 | Y |
| #ED visit | 1.24(1.17, 1.31) | <0.001 | Y |
| Abnormality of organs | 1.10(0.82, 1.47) | 0.52 | N |
| and/or soft tissues of | | | |
| pelvis affecting | | | |
| pregnancy | | | |
| Diastolic blood pressure | 1.09(1.02, 1.16) | 0.009 | N |
| in third trimester | | | |
| False labor at or after 37 | 0.65(0.37, 1.14) | 0.131 | N |
| completed weeks of | | | |
| gestation | | | |
| Asian | 0.40(0.32, 0.52) | <0.001 | N |

As can be seen from the data in Table 5, the largest contribution to PPD risk comes from just 8 factors: anxiety history, antidepressants, mood disorder history, depression in pregnancy, anxiety in pregnancy, mental disorder in pregnancy, and other disorder history. Together, these 8 factors have a total (nondimensional) weight of approximately 343, whereas all other factors combined have a total weight of approximately 42. In some embodiments, computational complexity may be reduced by considering only the largest contributing factors, and ignoring other factors. However, in other embodiments, greater accuracy or confidence (e.g., approximately 11% greater accuracy or confidence) may be achieved by considering all contributing factors. In still other embodiments, the weights of each contributing factor are selected or adjusted based on particular patient characteristics (e.g., age, race, etc.), or are calculated on a per-patient basis. Embodiments of the present disclosure may compute risk factor or treatment options based on any combination of the factors identified in Table 5, including some or all of the factors.

Furthermore, while most patient characteristics (e.g., mood disorders), other factors may be considered modifiable. Features are largely divided by healthcare use (medication prescription/ED visits) and diagnosis. For example, healthcare use may be considered modifiable, whereas diagnoses may be considered non-modifiable. Example modifiable characteristics include, but are not limited to, migraine disorder, use of beta blocking agents, delivery by cesarean section, use of direct-acting antiviral agents, use of antibacterial agents, and number of emergency department visits. In the example shown in Table 5, these factors account for a total weight of approximately 9, or 2.4% of the total risk. Thus, in some embodiments, a patient's PPD risk may be reduced by approximately 1-3% through behavior modification, which may for example be encouraged through social or psychological counseling.

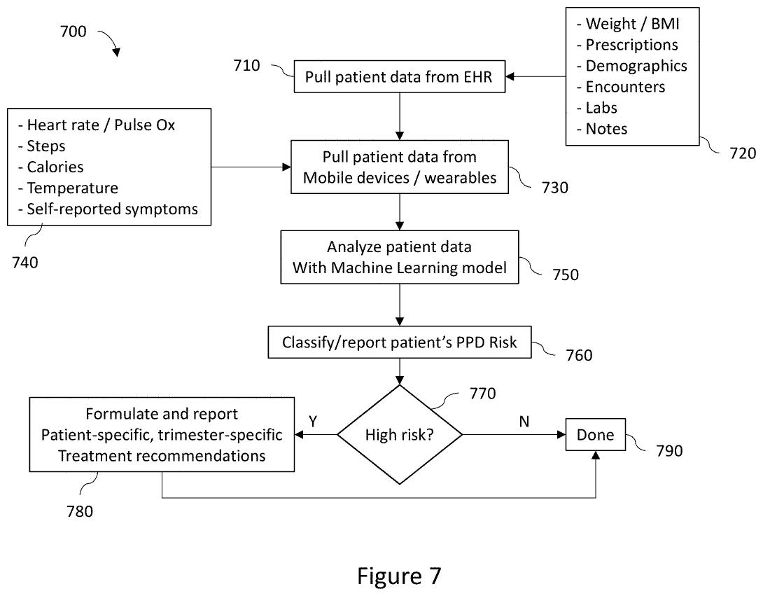

Example wearables may for example include the Oura Ring, Fitbit smartwatch, Apple Healthkit, and others. The PPD risk model 530 may for example be a machine learning system as described above in FIGS. 1-4 and below in FIG. 7.

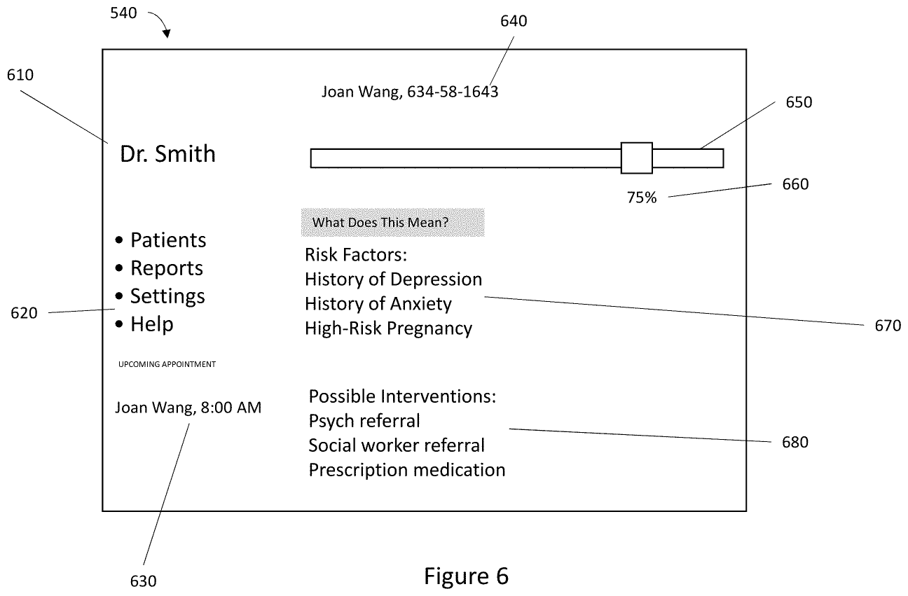

FIG. 6 shows an example clinical dashboard 540, in accordance with at least one embodiment of the present disclosure. In the example shown in FIG. 6, the clinical dashboard 540 includes a clinician identification area 610, options menu 620, next appointment reminder area 630, patient information area 640, risk score graphical representation 650, risk score numerical representation 660, risk factor detailed explanation area 670, and clinical actions recommendation area 680.

The clinician identification area 610 may for example include a name, title, and picture of the clinician running the dashboard application, and/or other information relevant to a medical practice. The options menu 620 may include various actions that can be taken by the clinician, including selecting a patient, selecting an action for the patient, adjusting settings, or seeking help. The next appointment reminder area 630 may for example include the name of the next upcoming patient and the time of the next upcoming appointment, or other information. The risk score graphical representation 650 may for example include a slider, graph, dial, or other graphical indication of a graphical risk score, relating to the patient's risk of developing postpartum depression, computed according to the methods described herein. The risk score numerical representation 660 may for example include ratio, percentage, or other numerical indication of the patient's risk of developing postpartum depression, computed according to the methods described herein. The risk factor detailed explanation area 670 may for example include a list of factors contributing to the risk score, including but not limited to psychological history, prescription medication history, OB-GYN history, and other factors. The clinical actions recommendation area 680 may for example include patient-specific, trimester-specific recommendations for clinical actions to be taken, determined according to the methods described herein. Such recommendations may be based on the patient's clinical history, the time since conception, the expected due date, and other factors, and may for example include prescription medications, referral to a social worker or mental health professional, additional testing, or other actions appropriate for limiting the risk of the patient developing postpartum depression. The clinical dashboard 540 presents information clearly and compactly, allowing the clinician to assess, at a glance, the patient's PPD risk, and any appropriate actions that may be taken to reduce the chance of PPD, or the severity of PPD if it should occur. Such information may, across a patient population, reduce the incidence and severity of PPD, and the associated social and financial costs. It is to be understood that the clinical dashboard 540 may show other data in other formats than shown here for FIG. 6.

FIG. 7 shows a flow diagram of an example clinical workflow method 700 in accordance with at least one embodiment of the present disclosure. It is understood that the steps of method 700 may be performed in a different order than shown in FIG. 7, additional steps can be provided before, during, and after the steps, and/or some of the steps described can be replaced or eliminated in other embodiments. One or more of steps of the method 700 can be carried by one or more devices and/or systems described herein, such as components of the processor circuit 850.

In step 710, the method 700 includes pulling patient data 720 from one or more electronic health records. Such data may for example include time since conception, expected delivery date, current or historical patient weight or BMI, prescriptions, patient demographics, patient encounters with the health care system (e.g., emergency department visits), lab results, and notes (e.g., marital status, socioeconomic status, or other information a clinician finds relevant to patient wellbeing), diagnoses, disability leave, discussions, and comments/paperwork.

In step 730, the method 700 includes pulling patient data 740 from patient-controlled devices, including but not limited to mobile devices (e.g., through a cell phone app), desktop or laptop computers (e.g., through an application or web portal), wearable devices, or other devices. Patient data 740 may for example include heart rate, pulse oxygen, daily step count, daily calories burned, temperature history, or self-reported symptoms such as cramps, fatigue, depression nausea, vomiting, changes in appetite, changes in bowel habits, vaginal bleeding, abdominal pain, sleep changes, increased urinal frequency, breathing problems, anxiety/worrying, psychosocial stressors, functioning, etc.

In step 750, patient data 720 and patient data 740 are fed into a machine learning (ML) algorithm (e.g., an algorithm designed and trained as described above, in FIGS. 1-4) configured to analyze the patient data according to the methods described herein. Example ML algorithms include, but are not limited to, logistic regression, support vector machine, decision tree, naïve Bayes, extreme gradient boosting, and random forest. Such ML systems share the traits of being high-performing (high sensitivity/precision) and interpretable, with a minimal number of features. Conversely, for some embodiments, black box models such as deep neural networks may be of lesser utility, as they have limited interpretability. For example, it is challenging for such systems to identify the top 5 predictors that contribute to individual predictions.

Missing data may for example be handled by multiple imputation (MICE). Sparse data may for example be handled by removing variables with low frequency and/or low associations with the outcome as judged by the assigned weight or odds ratio.

In step 760, the machine learning algorithm classifies and reports the patient's estimated risk of developing PPD, based on patient data 720 and 740.

The machine learning model training may for example be optimized using sequential forward selection (SFS)—a greedy search algorithm that searches for the combination of features that returns the maximum algorithm discriminatory power (Bradley, 1997). Starting with an empty feature set, SFS iteratively examines each feature combination such that the algorithm's performance can be maximized until the stopping criteria for the search is reached (T. and G., 2015).

Bradley, A. P., 1997. The use of the area under the ROC curve in the evaluation of machine learning algorithms. Pattern Recognition 30, 1145-1159.

T., L., G., T., 2015. SFS feature selection technique for multistage emotion recognition, 2015 IEEE 3rd Workshop on Advances in Information, Electronic and Electrical Engineering (AIEEE), pp. 1-4.

Algorithm parameters may for example be determined using a grid search for each algorithm that comprehensively searches for the best hyperparameters and parameters that result in the highest model performance as measured by area under the receiver operating characteristic curve (AUC)—pseudocode below. The stopping criteria for SFS may for example be defined as 1) no increase in the AUC by 0.001 after 10 consecutive iterations, or 2) the predetermined maximum number of feature set has been reached. SFS may for example be performed separately for women with, and without, mental health history to ensure that the model can predict for both types of patients when in actual use. Some embodiments may combine features selected from both SFS into a single feature set such that a single algorithm can be used for patients with and without a history of mental illness. Using the combined features, each of the machine learning algorithms may for example be trained using 5-fold cross-validation.

```
Input: K = {xᵢ,...,xₙ}
Output: Yₛ
Initialization: Y₀ = {∅}
for < i = 1,...,n > do
  x⁺ = arg max J(xᵢ + x), where x ∈ K - Yᵢ
  Yᵢ+1 = Yᵢ + x⁺
  if i ≠ 1 and (J₀ - Jᵢ) ≤ 0.001
  count = count +1
  if count =10, break
  else
  J₀ = Jᵢ, Yₛ = Yᵢ₊₁, count = 0
end for
```

Once the ML algorithm is trained, patient data (e.g., the data identified in Table 5) for an individual patient may then be used as inputs to the ML algorithm, to yield a total risk score from 0-1, or from 0% to 100%, reflecting the patient's estimated or predicted probability of having a diagnosis for postpartum depression within 12 months after delivery.

In step 760, the method determines whether the patient is high risk (e.g., whether a risk score determined by the machine learning algorithm exceeds a threshold value such as 50% or 75%). In some cases, a risk score may be a binary value (e.g., 1 or 0), a percentage (e.g., 0-100%), or an open-ended value (e.g., zero to infinity). A risk threshold may be determined separately from the risk score itself, and in some embodiments the threshold may be adjusted by the clinician as needed. Generally, there will not be a diagnosis attached to each risk level because the disease process likely has not yet started. Rather, the systems and methods disclosed herein are used to predict a likelihood of PPD disease before it occurs, so that interventive actions may be taken or planned to prevent the disease from occurring, or to limit its severity should it occur.

Preventive treatment (before symptoms develop) may be much more effective and efficient than interventional treatments (after symptoms develop), which are currently the standard of care. Unfortunately, the types of preventive treatments that are necessary and that have an evidence base to actually prevent postpartum depression (e.g. psychotherapy, medication) may not be feasible or appropriate to offer to the entire population of pregnant/postpartum women. Therefore, it may be important to accurately identify who is at increased risk and direct those resources accordingly. Currently there is no evidence-based or widely accepted tool in clinical practice to accurately identify who is at risk.

If the patient is not high risk, execution proceeds to step 790. If the patient is high risk, execution proceeds to step 780.

In step 780, the machine learning algorithm (or another algorithm such as a lookup table, expert system, statistical model, etc.) formulates and reports a list of recommended treatments. In some embodiments, treatment recommendations may be selected or modified based on patient-specific characteristics found in the patient data 720 and 740. In some embodiments, treatment recommendations may be selected or modified based on the trimester of pregnancy, or even the specific progress data of pregnancy. In some embodiments, treatment recommendations may be selected or modified based on a total PPD risk score of the patient. Thus, the treatment recommendations may be patient-specific, trimester-specific (or even date-specific in some embodiments), and/or risk specific. Execution then proceeds to step 790. An example treatment tree may look like this:

If no increased risk is present, then provide routine screening and management as per the provider's clinical assessment. However, if increased risk is present, then employ preventive intervention (dependent on drivers of risk). Preventive is considered ‘prior to the onset of psychiatric symptoms’, regardless of whether patient is pregnant or postpartum.

If mental health history is driving the risk: Assess for active symptoms/illness and address/refer as appropriate; Increase formal screening frequency; If stable on meds, engage in informed consent discussion about staying on meds throughout pregnancy/lactation; Make sure patient has an established relationship with mental health provider and coordinate care with that person; Refer for evidence-based preventive psychotherapy.

If medical/obstetric complications are driving the risk: Increase formal screening frequency; Increase appointment length or frequency (for obstetric appointments) to allow for additional counseling time about medical/obstetric conditions and psychoeducation; Where appropriate, refer to and coordinate with ancillary professional staff (e.g., nutritionist, doula); Assess social supports and counsel/involve them as appropriate; Consider supportive psychotherapy.

If social determinants of health (SDOH) are driving the risk: Assess for barriers to medical care; Engage patient in a discussion about strategies for meeting her maternal health needs (e.g., community support programs, transportation reimbursement, health insurance issues); Consider referral to social work; Consider referral for preventive psychotherapy; Increase formal screening frequency.

In step 790, the method is complete.

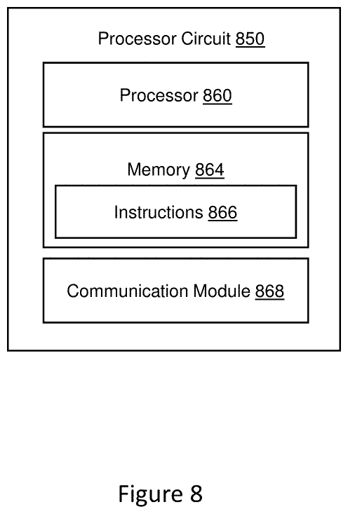

FIG. 8 is a schematic diagram of a processor circuit 850, according to embodiments of the present disclosure. The processor circuit 850 may be implemented on a mobile or desktop device, or other devices or workstations (e.g., third-party workstations, network routers, etc.), or on a cloud processor or other remote processing unit, as necessary to implement the method. As shown, the processor circuit 850 may include a processor 860, a memory 864, and a communication module 868. These elements may be in direct or indirect communication with each other, for example via one or more buses.

The processor 860 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 860 may also comprise another hardware device, a firmware device, or any combination thereof configured to perform the operations described herein. The processor 860 may also be implemented as a combination of computing devices, e.g., a combination of a DSP and a microprocessor, a plurality of microprocessors, one or more microprocessors in conjunction with a DSP core, or any other such configuration.

The memory 864 may include a cache memory (e.g., a cache memory of the processor 860), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 864 includes a non-transitory computer-readable medium. The memory 864 may store instructions 866. The instructions 866 may include instructions that, when executed by the processor 860, cause the processor 860 to perform the operations described herein. Instructions 866 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 868 can include any electronic circuitry and/or logic circuitry to facilitate direct or indirect communication of data between the processor circuit 850, and other processors or devices. In that regard, the communication module 868 can be an input/output (I/O) device. In some instances, the communication module 868 facilitates direct or indirect communication between various elements of the processor circuit 850 and/or the other computing systems. The communication module 868 may communicate within the processor circuit 850 through numerous methods or protocols. Serial communication protocols may include but are not limited to US SPI, I2C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to EHR retrieval, EHR storage, software updates, firmware updates, data sharing between the processor and an external server or device, or readings from wearables or other patient monitoring devices) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server over a wide area network (WAN), or over a local area network (LAN) with a local device such as a laptop, tablet, or handheld device, or may include a display capable of showing status variables and other information. Information may also be transferred on physical media such as a USB flash drive or memory stick.

A number of variations are possible on the examples and embodiments described above. For example, the methods described herein may be used to predict other postpartum health issues, including but not limited to anxiety, diabetes, preterm labor, postpartum hemorrhage, intrauterine growth retardation (IGUR), induction of labor (IOL), weight gain, hypertension/preeclampsia (HTN/PEC), thyroid disease, thrombosis/clotting disorders, or migraines.

Even with the best preventive treatments, there will still need to be interventional treatments. The devices, systems, and methods described herein may be used to improve interventional treatments as well, incorporating data from wearable devices and patient-reported symptoms to catch a developing depression in a more timely manner. Additionally, the method may be used for stratifying a patient's risk of increased morbidity and mortality associated with postpartum depression, which will facilitate efficient triaging of psychiatric referrals and emergency interventions.

The logical operations making up the embodiments of the technology described herein are referred to variously as operations, steps, objects, elements, components, or modules. It should be understood that these may occur or be performed in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the PPD prediction methods and systems. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the postpartum depression prediction methods and systems as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. A computer implemented method, comprising:
in a processor comprising a memory, performing the steps of:
identifying a patient population comprising a plurality of members;
collecting data from electronic health records of the plurality of members;
for each member of the plurality of members, identifying whether or not the member developed postpartum depression; and
training a machine learning algorithm to identify a patient's probability of developing postpartum depression using training data based on the collected data for the plurality of members.

2. The computer implemented method of claim 1, further comprising ranking selected elements of the collected data according to their statistical correlation with a patient developing postpartum depression.

3. The computer implemented method of claim 2, further comprising computing weights for each selected element of the ranked selected elements.

4. The computer implemented method of claim 3, further comprising removing elements from the training data whose weights are below a threshold value.

5. The computer implemented method of claim 2, wherein the selected elements of the collected data comprise socio-demographic data, medication data, lab data, emergency department visit data, marital status, mental health history, medical comorbidity diagnosis data, and obstetric complications.

6. The computer implemented method of claim 1, wherein the machine learning algorithm comprises at least one of a logistics regression, support vector machine, naïve Bayes, random forest, decision tree, extreme gradient boosting, or multi-layer perceptron classifier.

7. The computer implemented method if claim 1 wherein, when trained with the collected data, the machine learning algorithm has a Brier score of no more than 0.181 or an area under a receiver operating characteristic (ROC) curve of no less than 0.886.

8. A computer implemented method, comprising:
in a processor comprising a memory, performing the steps of:
receiving health data for a patient;
based on the health data for the patient, computing a risk score of the patient's risk of developing postpartum depression as a function of features selected from the health data for the patient; and
if the risk score exceeds a threshold value, computing treatment recommendations based on the health data for the patient; and
providing the treatment recommendations.

9. The computer implemented method of claim 8, wherein computing the risk score involves receiving at least some of the health data for the patient into a machine learning algorithm trained using health data from a population of pregnant women.

10. The computer implemented method of claim 9, wherein the machine learning algorithm comprises at least one of a logistics regression, support vector machine, naïve Bayes, random forest, decision tree, extreme gradient boosting, or multi-layer perceptron classifier.

11. The computer implemented method of claim 9, wherein the population of pregnant women includes at least some health or demographic characteristics in common with the patient.

12. The computer implemented method of claim 8, wherein the risk score comprises a numerical value between 0 and 1 or a percentage between 0% and 100%.

13. The computer implemented method of claim 8, wherein the computation of either the risk score or the treatment recommendations comprises a function of features selected from the health data for the patient, wherein the features are selected from anxiety history, use of antidepressants, mood disorder history, an indicator of whether there has been depression in pregnancy, an indicator of whether there has been anxiety in pregnancy, an indicator of whether there has been mental disorder in pregnancy, and a history of other disorders.

14. The computer implemented method of claim 8, wherein the computation of either the risk score or the treatment recommendations comprises a function of features selected from the health data for the patient, wherein the features are selected from palpitations, diarrhea, vomiting in pregnancy, hypertensive disorder, acute pharyngitis, hemorrhage in early pregnancy antepartum, patient's race is White, threatened miscarriage, abdominal pain, migraine, beta blocking agents, antihistamines for systemic use, hypothyroidism, placental infarct, patient's relationship status is single, deliveries by cesarean, direct acting antivirals, primigravida, pre-eclampsia, other antibacterials, number of emergency department visits, abnormality of organs and/or soft tissues of pelvis affecting pregnancy, diastolic blood pressure in third trimester, false labor at or after 37 completed weeks of gestation, or patient's race is Asian.

15. The computer implemented method of claim 8, wherein the patient's risk of developing postpartum depression comprises a risk of a diagnosis of depression within 12 months after delivery.

16. The computer implemented method of claim 8, wherein the threshold value is 50%.

17. The computer implemented method of claim 8, wherein the threshold value is 75%.

18. The computer implemented method of claim 8, wherein the health data is received from at least one of an electronic health record, a wearable device, a handheld device, or a census database.

19. The computer implemented method of claim 8, wherein the processor is a point-of-care processor.

20. The computer implemented method of claim 8, wherein the patient is a pregnant woman.
